(19) United States
(12) Patent Application Publication
(10) Pub. No.: US 2025/0389630 A1
(43) Pub. Date: Dec. 25, 2025

(54) AUTOMATED SURFACE TESTING DEVICE AND SYSTEM FOR USING SAME

(71) Applicant: Biomechanics Consulting and Research, LLC, Charlottesville, VA (US)

(72) Inventors: Jeff R. Crandall, Charlottesville, VA (US); Philipe C. F. Aldahir, Chattanooga, TN (US); E. Meade Spratley, Charlottesville, VA (US); Anna C. Workman, Charlottesville, VA (US); Alex M. Miles, Ruckersville, VA (US); Cody M. O'Cain, Charlottesville, VA (US)

(21) Appl. No.: 19/310,810

(22) Filed: Aug. 26, 2025

Related U.S. Application Data
(63) Continuation-in-part of application No. 18/075,952, filed on Dec. 6, 2022.
Application No. 18/075,952 is a continuation of application No. 17/509,422, filed on Oct. 25, 2021.
Application No. 17/509,422 is a division of application No. 17/192,752, filed on Mar. 4, 2021, now Pat. No. 11,154,244.
(60) Provisional application No. 62/985,126, filed on Mar. 4, 2020.

(51) Int. Cl. G01N 3/32; A61B 5/00; A61B 5/103; G01N 3/00; G01N 3/08; G01N 19/02

(52) U.S. Cl. CPC G01N 3/32; G01N 3/00; G01N 3/08; A61B 5/1038; A61B 5/6807; G01N 19/02

(57) ABSTRACT

A system to measure applied forces to surfaces allowing for safety assessment of apparel and surfaces, such as natural, artificial, hybrid, home, or commercial surfaces. The device and related system can compare deceleration test results to known results related to drop-mass and surface interaction, thereby allowing the system to closely mimic human behavior during movement on a surface.

### CROSS-REFERENCE TO RELATED APPLICATIONS

The present application is a continuation-in-part of and relies on the disclosures of and claims priority to and the benefit of the filing dates of U.S. application Ser. No. 18/075,952, filed Dec. 6, 2022, which claims priority to U.S. application Ser. No. 17/509,422, filed Oct. 25, 2021, which claims priority to U.S. application Ser. No. 17/192,752, filed Mar. 4, 2021, now U.S. Pat. No. 11,154,244, which claims priority to U.S. Provisional Application No. 62/985,126, filed Mar. 4, 2020. The disclosures of those applications are hereby incorporated by reference herein in their entireties.

### BACKGROUND

### Field of the Invention

The present invention relates to a, in aspects, mobile apparatus (the terms “apparatus” and “device” are used interchangeably in referring to the invention herein) and associated system that is completely or partially automated and is configured to measure surface (e.g., sport court) characteristics, and, in aspects, the safety and performance of athletic surfaces and/or apparel accurately and consistently in a drop-mass to surface interaction and/or an apparel-to-surface interaction, in a manner that is quantifiable and repeatable (e.g., shoe-surface interaction, ball-surface interaction, helmet-surface interaction, sporting equipment-surface interaction, apparel-surface interaction, object-surface interaction, and other interactions related to items that interact with a surface, such as, but not limited to, a playing surface for any sport). This application uses sport court(s) (e.g., an indoor basketball court) as an example but is not limited to sport court(s), because the invention can be used with sporting and non-sporting surfaces (e.g., concrete, asphalt, clay, earthen, ice, and/or hardwood surfaces) using a drop-mass, an end cap appendage attached to a drop-mass, instrumentation, and/or loads and motions particular to a certain sport (e.g., basketball, tennis, baseball, soccer, and so on). In examples only, a sport court can be considered to be a hard court surface and the immediate underlying environment, managed and prepared for fast and aggressive playing such as in basketball, soccer, field hockey, and volleyball. In aspects, the surface can be naturally or artificially grassed, or a mix of natural and artificial grass. Finally, in other aspects, the surface does not directly relate to sports, such as a grocery store or warehouse surface. With reliable game day, practice, or other data, causes and dangers of surface-related or shoe-surface related sports injuries on any surface can be better understood using particular appendages or impact interfaces (see, e.g., FIG. 8), also referred to herein as replaceable impact interfaces, that interact with the surface. Such understanding of the effect of the shoe-surface attributes as measured with this invention can help predict and mitigate injuries, as well as improve performance, through better surface and apparel characterization and rating(s). Moreover, as described herein, the drop-mass/surface interaction contributes to athletic performance, which, according to the present invention, could also be better characterized, understood, predicted, and even enhanced.

Accurately and consistently quantifying surface conditions, as well as the effect of surface conditions on athletic apparel in situations and conditions that mimic athletic movement, can be used to reduce athletic injury occurrences, improve player performance, establish accurate, independent standards for sports surfaces and non-sports surfaces manufacturing, installation, replacement, and maintenance, reduce unnecessary and expensive remediation and efficiently direct necessary rework, and can lead to standardizing surface assessments across sports facilities, including but not limited to soccer, tennis, basketball, baseball, volleyball, and golf, just to name a few examples, although the invention is not limited to athletic surfaces or apparel. With the instrumentation, system, products and methods described herein, these tests could be performed on clay surfaces (e.g., baseball skins, warning track, tennis courts), hard surfaces (e.g., tennis, pickleball, volleyball, basketball), sand surfaces (e.g., beach volleyball, bocce, horse tracks), ice surfaces (e.g., hockey), and other grassed (artificial, natural, or artificially reinforced) surfaces such as horse tracks, polo fields, and cricket fields/grounds. Such testing may also be used in non-athletic environments where synthetic or natural surfaces are utilized and where product longevity, safety, or performance can be enhanced through improved surface testing and surveillance (e.g. warehouses, industrial plants, dog parks, recreational areas, playgrounds, etc.).

### Description of Related Art

Available devices are capable of performing impact tests, including The Clegg Impact Soil Tester (CIST), the Advanced Artificial Athlete (AAA), and the USGA “True Firm” greens firmness tester. However, those listed above are insufficient biomechanical characterizations of a surface in one or more of the following: (i) the devices are not instrumented sufficiently to capture an acceleration impact history as biomechanics best practices require (for example, compliant with SAE-J211); (ii) the devices do not include realistic impact drop conditions for a given use, limiting the data to relative and subjective interpretation; and (iii) the devices are not able to collect biofidelic data for a variety of biomechanically informed conditions (e.g., different sports) due to the characteristics of its impacting interface and/or appendage. As an example, the Clegg Impact Soil Tester (CIST) is an existing device originally developed for measuring compaction of road base construction. It has been adopted for use in sports surfaces and used for measuring hardness attributes. It consists of a weighted cylinder equipped with an accelerometer dropped from a standard height through a guide tube perpendicular to the surface. A limitation in the current CIST form factor, among other things, is the lack of on-board electronics to allow for data collection of the entire time/acceleration history. Another limitation in the CIST device is the lack of player-based loading of the surface, contributing to a lack of sensitivity to important sports playing surface mechanical attributes that may affect the players, rendering the CIST not able to predict player surface safety as well as the invention described herein.

Other tests and test devices available on the market today have proven to be inconsistent, non-comprehensive, and subjective. Surface consistency, performance, and safety could be enhanced with the use of the novel device and method as described herein.

### SUMMARY

Due to the current limitations, it is an object of the current invention to allow for data collection on, for example, shoe-to-surface interaction while subject to athlete-derived and relevant applied forces and motions in order to properly correlate injury to surface characteristics, and/or characteristics of athletic apparel. In aspects, data collection might include, for example, kinetic and kinematic data during the shoe-surface interaction, measurement of surface characteristics through additional measuring devices, and recording of characteristics of the shoe and/or surface used for the shoe-surface interaction test. Because of the capabilities of the current invention, when injuries occur, the incidence of injury can be traced back to the co-localized surface data or apparel data collected by the inventive apparatus and compared amongst other cases of injury or, alternatively, with cases of non-injury; consequently, injury performance and metrics can be developed for sports surfaces, equipment, and apparel, and mechanical parameters measured can be used to develop safety and performance scores that can be incorporated during manufacturing of sport surface, equipment, and athletic apparel, thereby reducing injuries and increasing performance based on quantifiable data. In other words, once common injury metrics or injury risks are established, by way of example only, mechanical parameters prospectively measured can be minimized in the design and manufacture of surfaces or athletic apparel, thereby reducing injuries based on quantifiable data. To accomplish this, the current invention is, in aspects, mobile and completely-or partially-automated, and configured to provide reliable data that is repeatable and reproducible.

It is an object of the current invention to test the safety of hard courts and/or athletic apparel, especially shoes including but not limited to sports specific footwear, using a shoe-surface tester that determines and analyzes the mechanical interactions between shoes (or a foot-form) and an athletic playing surface and performs other measurements commonly taken on surfaces, and/or determines and analyzes whether the shoes and/or playing surface are up to standards and/or deemed safe for athletic events. The device simulates and measures shoe-to-surface interactions at loads and rates created or generated by athletes during performance up to and including those hypothesized or observed to be injurious. This involves measuring kinematics such as displacement, velocity, and acceleration components of the shoe and/or foot form moving in three dimensions (e.g., curvilinear, angled, and/or vertical), wherein, in aspects, the device is equipped with one or more accelerometer. In other envisioned embodiments, the drop mass can be dropped or otherwise forced in directions other than vertical, such as diagonal, angled, curvilinear, and/or horizontal, as well as having the drop mass and/or device work in rotation, and any combination of these forces at the same or different times. Beyond kinematics, the apparatus may apply and measure all components (dx, dy, dz, rx, ry, rz) of six degrees of freedom kinetics, such as forces and moments, of the shoe, foot form, drop mass, or surface interface. These potential forces, directions, and rotations, are explained in more detail in U.S. Pat. No. 11,154,244, which is hereby incorporated herein by reference.

The device may use a drop-mass (e.g., a foot-form or appendage, by way or example) connected to a system of nested frames or a Stewart platform to accomplish this. In a preferred embodiment, the device comprises a drop mass that is tuned or optimized to stress the playing surface like a player does by faithfully reproducing shoe-surface pressure and depth of penetration. Moreover, the appendage drop-mass (e.g., foot-form) can be interchangeable (see, e.g., FIG. 8), meaning, in aspects, one appendage can be removed and replaced with another one, such as a different shape, material, stiffness, etc. For example, the drop-mass can be configured to attach and detach different replaceable impact interfaces (also referred to herein as, for example, appendages, end-caps, or end-cap appendages, replaceable end-caps, and variations thereof), wherein those replaceable impact interfaces are what substantially interact and/or interface with a surface during testing, for example a shoe, a ball, a helmet, or another piece of sports apparel. Replacing the end-caps may also alter the weight of the mass to match specific conditions depending on the sport or activity in question.

Thus, the end cap appendage can, in aspects, be actuated through its prescribed load or positional path by a mechanism capable of imparting and withstanding the significant forces and moments without unwanted mechanical deformation, friction, or fatigue that might otherwise influence the data collected. In a preferred embodiment, the device is not actuated, but rather the drop-mass appendage is dropped and gravity “pulls” the appendage downwards to the playing surface; in other words, a dropped mass set is dropped from a prescribed height that impacts the surface with a (certain) approaching velocity resulting in X joules of energy deposited into the surface (e.g., the stress), and the measurements reflect what happens to that energy (e.g., how hard it hits, how fast force and pressure dissipates, how much of the energy is returned to the mass, how deep it penetrates and/or slides, etc.). Based on those measurements, as explained herein, a user of the device (in some cases via one or more processor, one or more controller, and/or one or more sensor) can determine properties of the surface-end-cap interaction. In embodiments, the device may also automate or assist with:
a) Measurement of Energy absorption and rebound/return through measurement of acceleration of a mass or mass-spring system dropped onto the surface, through an on-board data collection system, and reporting of this data to the user.
b) Measurement of surface hardness (e.g., Head Injury Criterion measure; peak g acceleration metric), using devices specified in ASTM F1702 and/or ASTM F355 or other drop test standards, collected through an on-board data collection system, and reporting of this data to the user.
c) Surface depth measurement (e.g., synthetic turf granular infill depth and/or evenness);
d) Detection of surface moisture levels;
e) Detection of surface finish roughness levels;
f) Measurement of environmental factors, such as air temperature and pressure, ground temperature, air humidity, or other factors; and/or
g) Characterization of surface maintenance, such as through flagging undesirable testing results.

It is a further object of the current invention to fully characterize a sporting surface in a way sufficient to direct changes to the surface and/or apparel/equipment for improved performance and/or injury prevention. This mechanism will incorporate all or part of the tests in a controlled manner. By actuating test modes, the system can rely on the data being consistent across locations, facilities, and operators, as human-to-human variability inherent in testing with manually powered devices is removed from the process. For example, the system may automatically set the mass drop height and/or angle. The system may further automatically actuate the release mechanism to minimize human interference in the collected measurements. The system may also incorporate locational measurements of the top surface of the test surface into test actuation or data processing. To maintain safety, the system may display the current “state” of the system (for example, whether the system is safe for manual operations or ready to complete a test). Finally, the device can utilize a variety of impacting appendages to replicate a specific test condition. For example, when testing focuses on lower extremities, the appendage might use shoes or shoe soles that can be released/propelled onto the playing surface. When testing focuses on concussions (head-to-surface), the appendage might be an oblong-shape similar to a bicycle or hockey helmet.

The appendage can be informed by an athletes' or another sporting equipment/apparatus' kinetics and kinematics, and is interchangeable for customizability.

In embodiments, in regards to the form factor, the drop tester as described herein is biomechanically informed by significant research, trial-and-error testing, and engineering optimization to mimic athlete player loading (e.g., vertical and/or shear forces imparted by an athlete). The result of that research is a novel impact mass, impact interface shape, and impact velocity incorporated into the device as described herein.

Additionally, the device and system described herein provide processing of data collected by the device after use.

### BRIEF DESCRIPTION OF THE DRAWINGS

The accompanying drawings illustrate certain aspects of embodiments of the present invention and should not be used to limit the invention. Together with the written description the drawings explain certain principles of the invention.

FIG. 1A and FIG. 1B depict potential embodiments of the apparatus for testing interaction(s) of athletic apparel, athletic equipment, shoes, or other objects, with a surface, according to embodiments of the present invention as described herein.

FIG. 2 depicts a potential embodiment of the apparatus for testing interaction(s) of athletic apparel, athletic equipment, shoes, or other objects, with a surface, according to embodiments of the present invention as described herein.

FIG. 3 depicts a flow chart/workflow associated with the apparatus for testing interaction(s) of athletic apparel, athletic equipment, shoes, or other objects, with a surface, according to embodiments of the present invention as described herein.

FIG. 4 depicts a potential embodiment of circuitry and related aspects associated with the apparatus for testing interaction(s) of athletic apparel, athletic equipment, shoes, or other objects, with a surface, according to embodiments of the present invention as described herein.

FIG. 5A, FIG. 5B, and FIG. 5C, show charts related to testing and using the invention as described herein, including testing a bouncing basketball, a cutting/lateral movement of a player on a basketball playing surface, and a summary of the test conditions.

FIG. 6 shows an example related to testing and using the apparatus according to embodiments of the present invention as described herein, tested against currently-available technology.

FIG. 7 depicts predicted testing results/data related to testing and using the apparatus, according to embodiments of the present invention as described herein.

FIG. 8 depicts different potential replaceable impact interfaces, according to embodiments of the present invention as described herein.

### DETAILED DESCRIPTION OF INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

In embodiments, the device described herein can collect high-resolution, low-noise data from an accelerometer, for example, and upload the data to the device, another electronic device associated with the device, and/or a cloud-based server over, e.g., Wi-Fi. Optionally, the device has wheels or removable wheels for ease of operation, an automated drop mechanism to increase data quality, and a user interface. The user interface, in aspects, can be a color digital display and an operated by means of a touch screen or physical controls (e.g., knob with rotary encoder). In aspects when using the device, the user will start the device which will then initialize or boot up the data processing unit, verify correct and accurate function of some or all the sensors, and display a main menu of selectable options to a user. In embodiments, a user interface can include a display and control mechanism(s) of the apparatus for testing interaction(s) of athletic apparel, athletic equipment, shoes, or other objects, with a surface. In aspects, the user can interact with the display, such as by analog or electronic/digital buttons, levers, keys, or knobs, by way of example only. In aspects according to the current invention herein, certain assemblies can be three-dimensionally (3D) printed.

In embodiments, the device is a hard-court surface characterization or a shoe-surface characterization (SSC) device to mimic shoe-surface interaction. The device can enable the fidelic and accurate computation modeling of a playing surface (e.g., boundary conditions for athletic play), such as a hard court, or artificial or natural turf, as a tool to assess and optimize surface performance, and ultimately to reduce injury. The apparatus and associated system can impose a constant and/or dynamic or variable vertical force or force (or force(s) at varying angles, which can be adjusted) on the drop-mass assembly while collecting motion profile data on the drop mass as it engages and releases from the surface. This data can be used to identify the differences in quality and/or consistency of playing surfaces, and the quality of athletic apparel, during injury (or non-injury) compared to the characteristics different sporting surface designs, non-injury situations, and/or safer or less safe athletic apparel. In addition to data collected during the drop mass interaction test, other characteristics of the surface tested can be saved and linked with the test data. In some embodiments, the device can apply horizontal force(s), diagonal force(s), and/or rotational torque, which can indicate the amount of traction or friction (or lack thereof) of a shoe or other apparel/equipment. In aspects, the device can be used to test non-athletic playing surfaces, such as home or commercial flooring. In aspects, the device can be used to test interaction of items that are not shoe or shoe-related, such as a helmets, balls, hockey sticks, golf club heads, and other items that may interact with a surface.

The system may also apply and measure admittance, therein applying a force/torque and measuring displacement or velocity; thus, the system may be configured to apply a particular force/torque in order to measure the impact on, for example, an athletic shoe, a human body or body part, and/or a playing surface.

In aspects, the current invention tests mechanical interactions between the drop mass-to-surface interface using a vertical force, and motion data is recorded via data acquisition, and therefore the system is capable of recording accurate and repeatable results. In aspects, the apparatus is configured to collect in-situ displacement, velocity, force and torque data, record impact hardness, measure infill depth (turf), moisture content (grass), and/or upload or download data manually or automatically.

In aspects, the apparatus processes and displays data tailored for a certain target audience. In aspects, there might be some “hard-coded” data with some data analysis built in locally, remotely, or on a server. In another example, the analysis may be performed online via cloud or remote manipulation of test data and metadata stored in a server.

The system can also run on-board diagnostics tests on each drop test, as, for example, a means of verifying data quality. As part of the data QA/QC procedure, the system can perform on-board calculations, alert the user of non-compliant sensors, identify a bad test, and prompt the user to retest or service the device.

The system can be capable of objectively and quantitatively scoring surfaces, and evaluating geographical compliance of a surface with a standard or protocol (using, for example, a local positioning system (LPS), a “hard-coded” location system, or a global positioning system (GPS) to evaluate an entire playing area or a portion of a playing area and recommending localized intervention/maintenance).

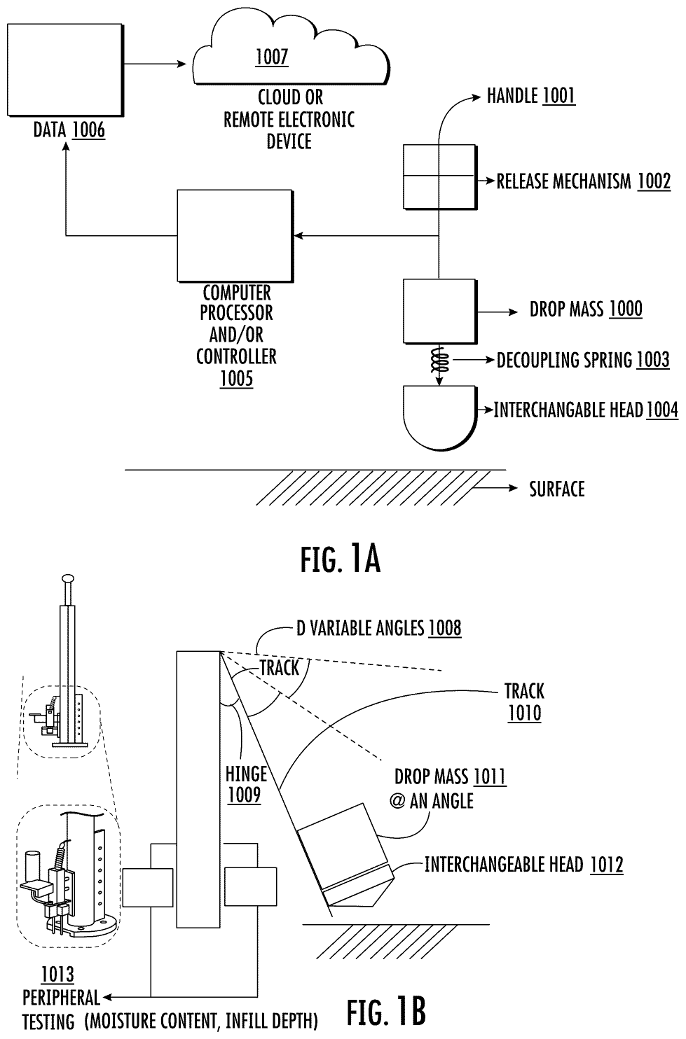

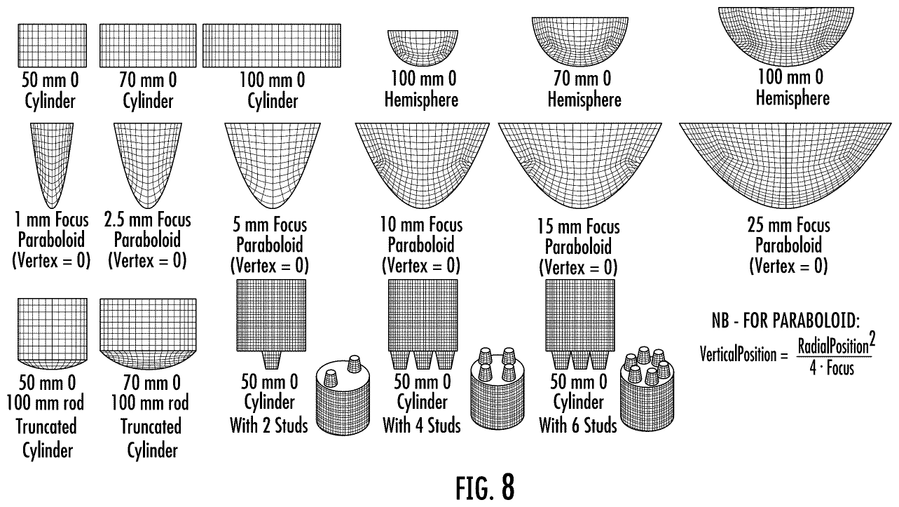

Turning to the figures, FIG. 1A and FIG. 1B show embodiments of the current invention. In aspects, attached to an elongated substantially vertical pole, tube, shaft, track, or other structure, are the electronics and dropping mechanism for dropping the drop-mass onto the playing surface (e.g., hard court). In embodiments as shown in FIG. 1A, the device may comprise a drop mass 1000, one or more parts for associated electronics, a handle 1001, a release mechanism 1002, a de-coupling spring 1003, and replaceable, interchangeable heads 1004, which will interact with the surface (see, FIG. 8 for more detail). In embodiments, the device will interact with a computer/computer processor and/or controller 1005, which can send and receive data and instructions 1006, such as to and from the cloud, a server, or another computer 1007. FIG. 1B shows an embodiment allowing for multiple dropping angles 1008, which can generate shear force(s) to test, predict, or otherwise analyze, the traction of, for example, a shoe on a playing surface, or friction or other forces indicative of, for example, a helmet hitting and sliding across a surface, such as a road or indoor track. In embodiments, the device will comprise a hinge 1009 and a track 1010 (to which the drop-mass 1011 can be attached or connected to), allowing for variable angles to drop the drop mass. This allows the drop mass to be dropped at an angle, if preferred, and thus, in aspects, the device is not limited to vertical dropping. In aspects, the angle can be adjusted manually or automatically. This embodiment can also include replaceable, interchangeable end-cap appendages 1012. The embodiment also shows peripheral sensors and ways of measurement for additional functionality, such as testing for surface moisture content, infill depth, gloss levels, scratching resistance, and other qualities or quantities related to the surface, by way of example, as explained herein 1013. In aspects, upon instructions by a user, the drop-mass will drop and collect data. The system can be trained to generate a training model that can analyze qualities of the playing surface and compare that data to the training model. The training model can be trained using, by way of example, analysis or analyses of sports apparel interacting with a similar or the same type of playing surface. (Non-athletic applications are also envisioned, as explained herein.) Based on known, a priori data, such as athlete-surface interaction, a user can compare the results of the device's test drop and thereby predict whether the surface is safe, not safe, liked, or disliked by players, or some other quality or property the user wishes to analyze. The training model can also be trained using data collected from the same or a similar device being used on a selected or known surface. For example, the training model can be trained using the device on a safe playing surface. If the device on a field or playing surface to be tested is used and collects results similar to what would be expected of a safe playing surface based on past testing of safe playing surfaces, the system will inform the user that the tested playing surface is safe (or degrees of safe). On the other hand, if the device is trained on an unsafe playing surface, results using the device on a field to be tested that collects data similar to what was experienced when training on unsafe surfaces, the system will inform the user that the testing surface is unsafe (or degrees of unsafe). This is true for other surfaces and surfaces interactions, too. The model can be trained using the same or a similar device. It can also be training using a real test subject. For example, a user can employ a drop test, and the interaction on different surfaces (e.g., from safe to unsafe) can be recorded and then used to compare against results on surfaces to be tested by the device described herein.

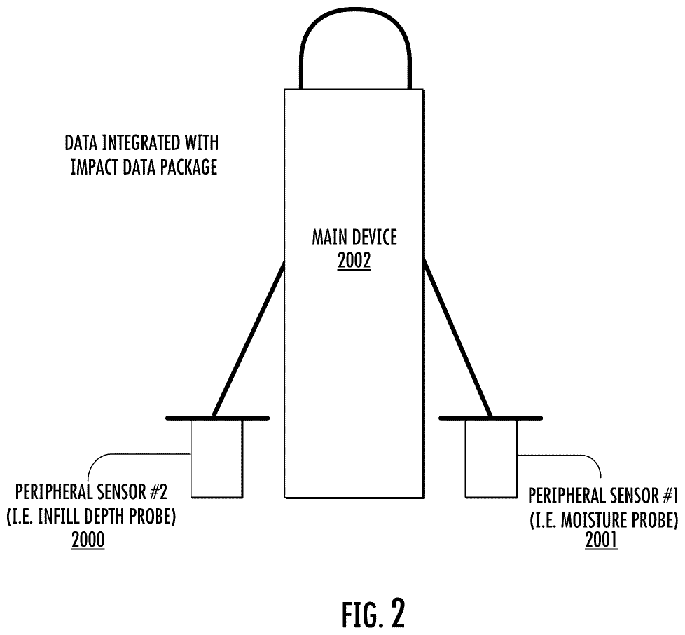

Further according to FIG. 1A and FIG. 1B and embodiments described herein, the ability for an angled drop allows for the ability for shear measurement (e.g., that could be used as a proxy for traction, for example). The vertical drop can provide a normal component. In aspects, integrated peripheral sensors are included along with the device. The measurements can be taken alongside impact tests and used to contextualize the impact testing results. As shown in FIG. 2, for example, sensors may be incorporated in, on, or near, the device 2002 itself, allowing for automated or semi-automated collection of, by way of example, infill depth (synthetic turf) 2000 and/or surface moisture (natural and hybrid grass) 2001 on the same data package as the impact data. Although it is also envisioned that peripheral sensors may be located remote from the device itself. Further, the interchangeable impact head design that couples to the drop mass can allow for measurement of multiple sports surfaces (beyond grass and turf), if desired. FIG. 8 shows examples of the replaceable impact interfaces.

The device can include a display where results can be shown to a user, such as a touchscreen display. In aspects, output from the test can be shown on a display, wherein the display is on or otherwise attached to the device. In other embodiments, data from the device can be sent to another electronic device, such as a phone, tablet computer, computer, laptop computer, server, cloud, or internet, by way of example only. In aspects of the current invention, some or all of the data collection, processing, streaming, and/or analyses can be performed and/or stored in the cloud. As a result, in addition to onboard result summaries, users have the ability to view data and analyze insights (in some cases, immediately) after testing through, for example, a web-based portal. In aspects, heat maps, data summaries, and trends, can be available immediately post testing, given that the data is uploaded by the user.

In embodiments, one or more human is tested in a laboratory or other setting, for example an athlete will shod athletic footwear and interact with a playing surface and kinetics and kinematics data is recorded as part of the training model. In aspects, those results are digitized through finite element (FE) modeling, such as surface FE+shoe FE+player data. Also, test conditions, such as energy, depth of engagement, and pressure, can also be measured and modeled. This data can be used to train the training model and therefore be used to generate testing insights from in-situ use. In other words, that training model can be used to interpret data recorded from the device when being used to test a sports surface, thereby the training model is predictive in nature, which can lead to machine learning and artificial intelligence capabilities and applications according to the system described herein.

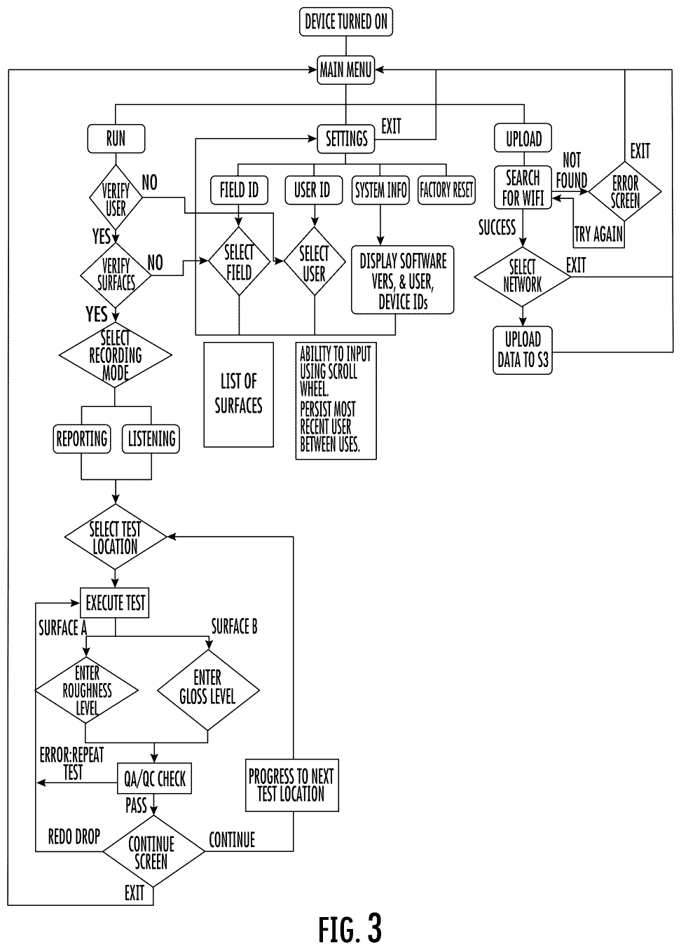

FIG. 3 shows an example of a workflow according to the current invention. For example, a user/operator may start the system by signing in, and then the user may, in embodiments, choose a full matrix field check, including the location. The user may also or alternatively choose to check the system, such as a level check, upload data, checking the system configuration, choosing a factory reset, a system information check, a configuration update, and/or an error messages check. In aspects, the user may choose a single drop test, and may choose or input such information as the facility or location, the setup and teardown for a drop, and details for the drop. In aspects, there may be advanced settings, such as changing advanced configuration values, changing specifications, changing drop timing and/or drop centering, adjusting the drop-mass, adjusting a track/shaft or drop-mass angle, changing the rest position of the drop-mass, changing the drop curve, changing the pulled position of the drop servo, and/or changing an acceptable tilt range for the level sensor.

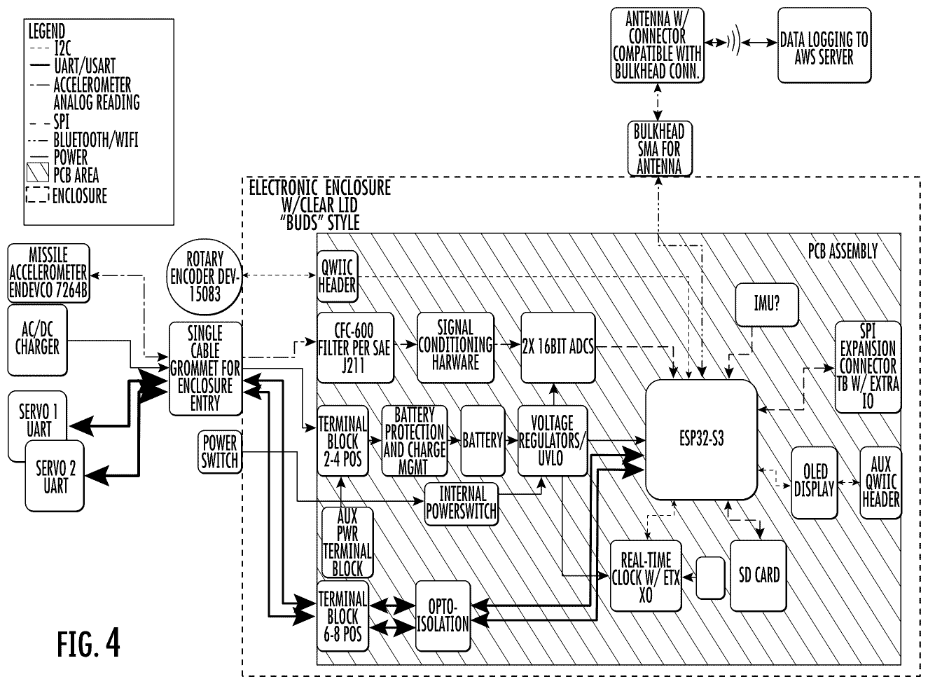

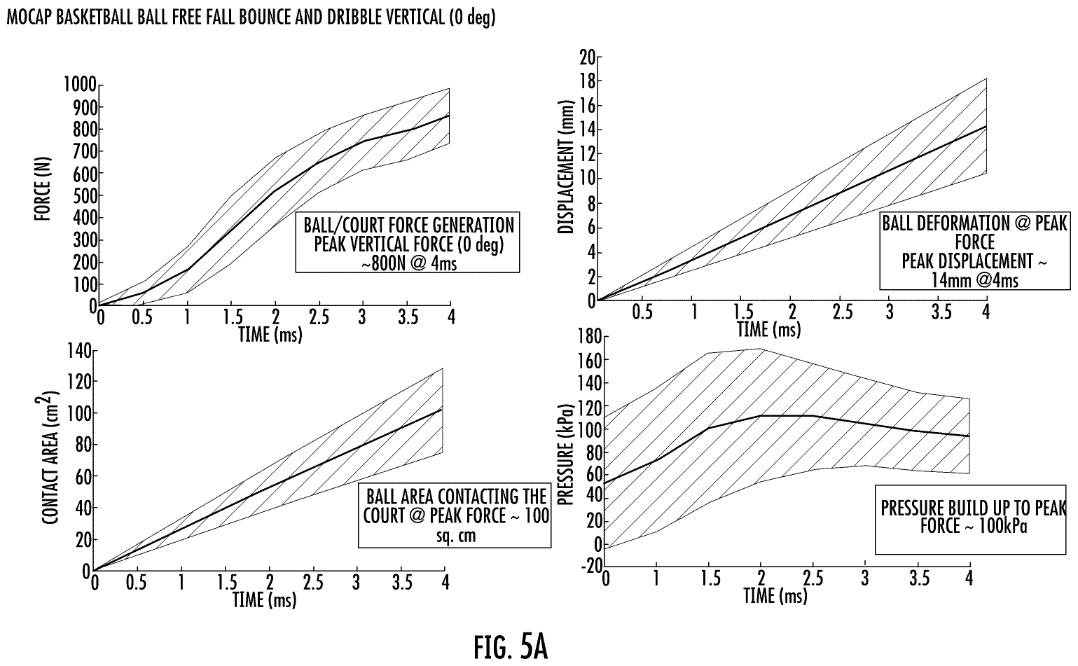

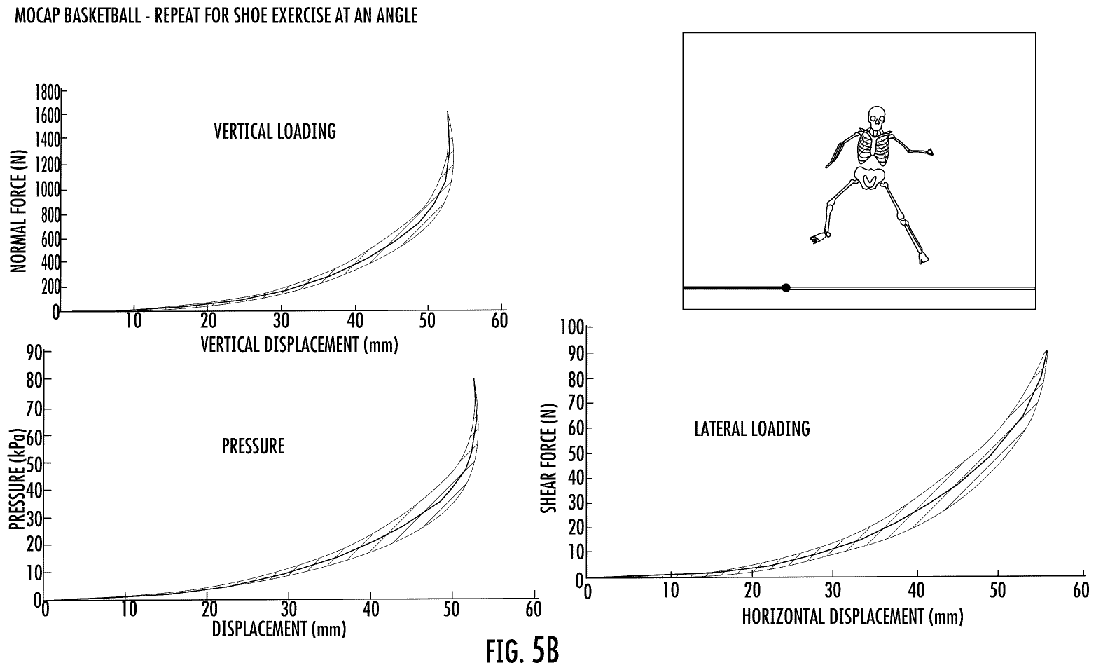

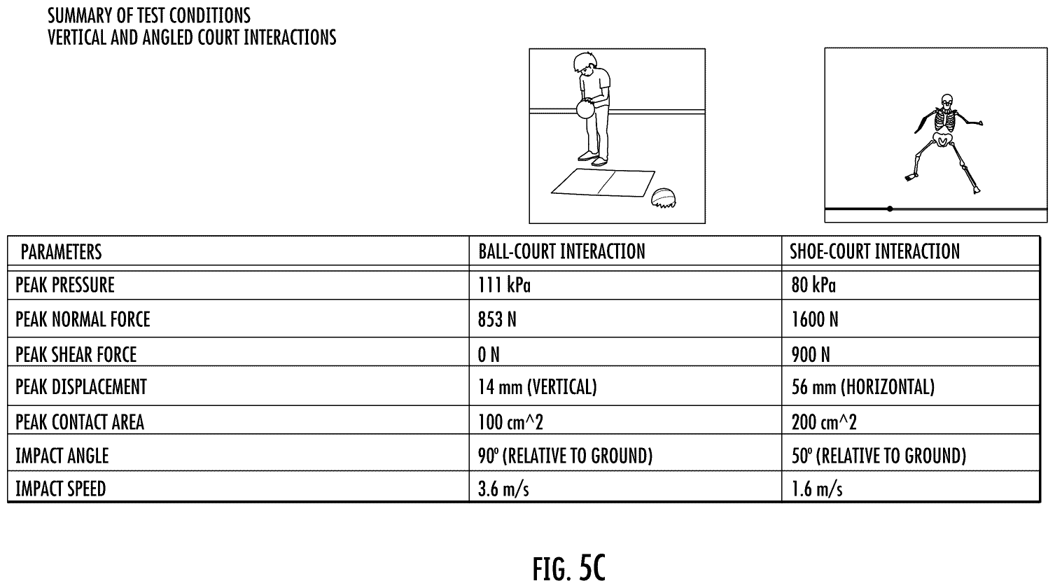

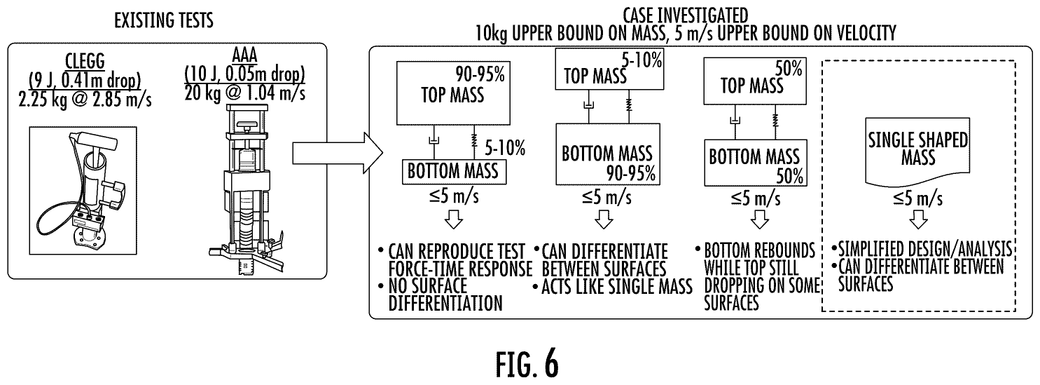

FIG. 4 shows an example of electronic circuity used to operate the drop mechanism and record data. FIG. 5 shows testing according to testing of the invention described herein. FIG. 6 shows additional testing results as described herein.

In aspects of the drop-mass assembly, it may include a spool drive pulley and/or a shaft spool pulley and/or a shaft cable pulley, a shaft assembly, a spool clutch, a spool gear motor (such as a servo motor), a rotation lockout solenoid, an actuator, a load cell, and an accelerometer. In aspects, the system allows for real-time or near-real-time computer-mediated adjustments to the device actuation in response to the loads/moments perceived at the drop-mass and surface interface, or other feedback. Real-time can mean results within seconds or milliseconds.

In aspects, the invention herein is a device to enable the fidelic and accurate

computation modeling of object-surface interaction in sports, as a design tool to assess and optimize surface performance and/or to reduce player injury.

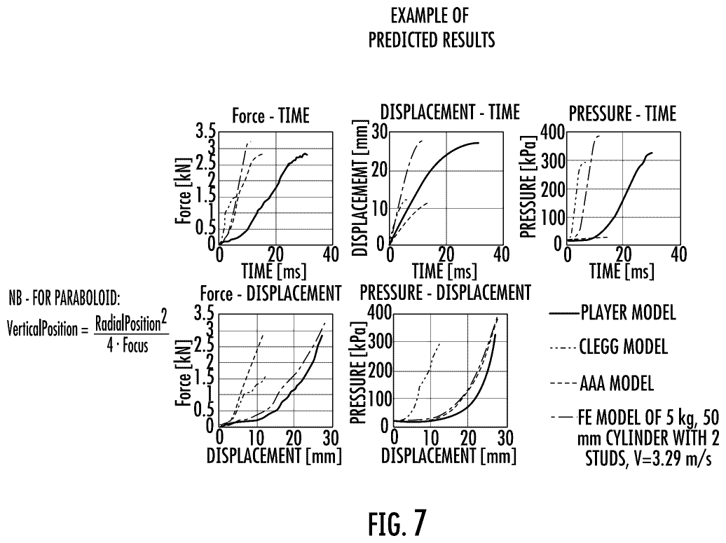

As shown in FIG. 7, investigation using FE modeling tested various impact interfaces weighing 2.5 kg, 5 kg, and 10 kg masses; impact velocities of 1.25 m/s, 2.5 m/s, and 5 m/s; and numerous impactor geometries. Results of all models were predicted, plus iterations on selected designs were also assessed. This trial and error, among other things explained herein, helped lead to currently proposed and claimed design parameters for the missile/foot form and properties in the way the drop mechanism drops its missile according to embodiments of the current invention. FIG. 8 shows different end-caps that can be used on the drop-mass.

Different end-caps, or impactor geometries, can be used on the drop-mass, and switched in and out depending on the test results desired. For example, for a certain kind of shoe, one type of end-cap can be used on the drop-mass, whereas a different type of shoe would achieve better results if a different type of end-cap, such as one mimicking the materials of that particular shoe, were used on the machine. The device is not limited to footwear. For example, if head-to-surface interactions were desired to be measured, an oblong end-cap could be attached to the drop-mass, thereby mimicking a helmet. This could also be true for golf club heads and any other physical item that interacts with a surface, such as a playing field surface. By way of example only, in an embodiment for testing a golf swing or any other swinging motion, the drop-mass can comprise a swinging apparatus, such as a swinging pendulum, whereby instead of being dropped, the drop-mass or appendage can be swung.

In summary of the design parameters, a single mass indenter geometry, according to the current invention, can differentiate between different surface constructions. The foot-form and/or drop mass according to the current invention can, in some cases, meet the design target for force and a pressure-displacement response under impactor situations that are similar to a shoe or athletic apparel being worn by an actual athlete (or other apparel/equipment worn by a non-athlete).

In embodiments, the device can comprise a data port, computer processor, antenna, memory storage unit, receiver, transmitter, controller, battery, charger, charging port, and other electrical components. In aspects, the apparatus may include global positioning systems (GPS), local positioning systems (LPS), or other devices to, for example, register its position relative to the field or surface being tested. The apparatus may comprise a data acquisition system (DAQ), camera(s), actuator drivers, and/or control unit. In embodiments, the apparatus will not only test shoe and surface interactions, but can also include sensors to test the surface conditions, such as a surface impact hardness sensor and a moisture sensor, and/or an infill depth probe. Regarding the shoe-surface testing aspects, the apparatus may also comprise a test shoe displacement sensor, shoe actuator, instrumented insoles capable of measuring force and pressure, vertical preload actuator, adjustment mechanism, a test shoe wrench sensor (such as a multi-axis load cell or multiple single axis load cells), an attachment mechanism (such as a plate), and a foot-form and/or a test shoe for example on a foot-form. The apparatus may include the camera(s) attached to the apparatus or employ a drone to hover above the surface for visual inspection.

As shown in FIG. 5A, FIG. 5B, and FIG. 5C, and according to embodiments of the present invention, real world testing results can be used to train the system parameters, including simulating a bouncing basketball, a cutting/lateral movement of a player on a basketball playing surface, and the test conditions can be summarized. For example, as shown in FIG. 5A, a basketball can be used on a hard surface and preferable results can be recorded to inform the system of, for example, how an optimal basketball surface should react upon interacting with a dribbled basketball. Similarly, a real world player can be recorded doing basketball moves on a basketball surface to inform the system of preferable surface conditions. (See, FIG. 5B.) These results can then be summarized and inputted into the system for later comparison purposes, by way of example. (See, FIG. 5C.)

Testing can be performed in a single test location and the surface can be tested until all of the tests are completed. In an example, several tests can be performed to generate results, and the user keeps moving around a field until all chosen locations on the field are completed. Single location testing could be testing at a single site, and may provide options for a user to redo, move on, or quit after each test, by way of example.

In embodiments of system architecture according to the invention described herein, the system may include a USB charge/program and AWS AIP (WiFi). In embodiments, it will include a processor and storage, battery, charger, microchips, WiFi chip, Bluetooth chip, accelerometer, clock, sensor for leveling the device, a display, and/or a drop servo motor.

### EXAMPLES

### Metadata Collection

In aspects, the device and system can use metadata to assist with generating test results and other information related to the testing. For example, metadata can include data about the data for a drop (or more than one drop) of the drop-mass. In aspects, the metadata can include scaling factors used to scale the data to g-forces. It can include who was the user (e.g., operator). It can include what kind of surface is being tested. In aspects, metadata items recorded by the system can include but are not limited to:
Location/facility/surface name: Selected testing location
User: Selected user conducting the testing
Position: facility/location coordinate for each drop test
Scaling and transducer sensitivity factors: sig_to_mV, mV_to_g for the accelerometer scaling and conversion to appropriate engineering units.
Device Identification: for example, different devices may each have their own unique identification (ID)
Peak G acceleration(s) and/or g force(s): Output of the filtered peak-g-forces calculation
Frequency: The sampling and output frequency of the analogue to digital converter (ADC)
Level Threshold:
   X and Y angle of the levelness according to the IMU
   Calculated absolute level from ground
   Level threshold limits.
Real-time clock time and timestamp
WiFi™ and/or Bluetooth™ Configuration: Network used for upload and whether it was secure.

### Additional Testing

A test was run using a full-factorial analysis looking at one or more effect of one or more of:
Impactor diameter—50 mm and 70 mm
Impactor face radius—0 mm (cylinder), ½Ø (i.e., both 25 mm and 35 mm), and 50 mm
Initial impactor velocity—1.25 m/s, 2.5 m/s, and 5 m/s
Impactor mass—2.5 kg, 5 kg, and 10 kg

Force—Time Test Results—These results showed actual results from athlete testing (lateral shuffle test) vs. the two devices commonly used today (CIST and AAA) vs. various models for different impact interface geometries and boundary conditions (impact velocity). These results represent an optimization step towards determining test conditions such as mass, angle of impact, impact interface geometry, and impact velocity, needed to better match the players loading of the surface.

Displacement—Time Test Results—These results were shown to be similar to Force—Time Results, but on a displacement vs. time basis, rather than force vs. time.

Pressure—Time Test Results—These results are similar to Force—Time Results, but on a pressure vs. time basis.

Force—Displacement Test Results—These results are similar to Force—Time Results, but on a force vs. displacement basis.

Pressure—Displacement Test Results—These results are similar to Force—Time Results, but on a pressure vs. displacement basis. In aspects, this condition is favored for a match with the device design for two reasons: (i) to maintain the engagement seen on the V-cut drills (cleats penetrating up to 30 mm in the pitch surface) and (ii) to match the pressure or stress/strain properties put on the surface rather than the force, which can allow for a reduction in device size and complexity. This is novel, as (i) most devices do not interrogate the depth of the surface (most have flat end impactors), (ii) most devices do not match any player-level stress applied to the surface, and (iii) most impactors have too short of a pulse compared to the player engagement (the studded endcap design lengthens the pulse by gradually engaging/penetrating into the surface). These are problems that can be solved by the present invention described herein.

Embodiments of the invention include a computer readable medium comprising one or more computer files comprising a set of computer-executable instructions for performing one or more of the calculations, steps, processes and operations described and/or depicted herein. In exemplary embodiments, the files may be stored contiguously or non-contiguously on the computer-readable medium. In aspects, the files or data may be sent directly or indirectly to the cloud or remote servers(s). Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers or involve a network of remote servers hosted on the internet. In aspects, local, edge, or remote computing possibilities are used to store, manage, and process data. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general-purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes, and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A system comprising:
one or more processors, one or more controllers, or both one or more processors and one or more controllers;
a drop assembly comprising a drop-mass connected to a track, wherein the drop-mass is configured for attachment and detachment of one or more replaceable impact interfaces, and wherein the track is adjustable to change an angle at which the drop-mass is dropped;
one or more sensors;
wherein the drop-mass, with or without a replaceable impact interface attached to the drop-mass, can be dropped onto a surface in one or more of a vertical orientation, a substantially vertical orientation, an angled orientation, or a diagonal orientation, thereby generating one or more of, deceleration data test results, force data test results, motion data test results, or acceleration data test results;
wherein the one or more processors, the one or more controllers, or both the one or more processors and the one or more controllers, are operative to calculate the one or more of, deceleration data test results, force data test results, motion data test results, or acceleration data test results;
wherein those one or more of, deceleration data test results, force data test results, motion data test results, or acceleration data test results, are compared against known deceleration, force, motion, or acceleration data related to one or more surface interactions; and
wherein, based on the comparison between (a) the one or more of, deceleration data test results, force data test results, motion data test results, or acceleration data test results, and (b) the known deceleration, force, motion, or acceleration data, the system outputs one or more of: suggested adjustments to the drop-mass, suggested adjustments to the drop-mass and surface interaction, suggested adjustments to the angle of the track, suggested adjustments to the one or more replaceable impact interfaces, an analysis of a safety of the surface, an analysis of a safety of an object or a simulated object interacting with the surface, or combinations thereof.

2. The system of claim 1, wherein the known deceleration, force, motion, or acceleration data, is completely or partially based on data resulting from one or more human interacting with a same or a similar surface as a surface being tested or analyzed using the system.

3. The system of claim 1, wherein a tunability of a surface engagement is informed by test results from one or more human interacting with a same or a similar surface as a surface being tested or analyzed using the system.

4. The system of claim 1, wherein comparing (a) the one or more of, deceleration data test results, force data test results, motion data test results, or acceleration data test results, and (b) the known deceleration, force, motion, or acceleration data, allows for the system to evaluate safety and/or performance of one or more of athletic apparel, athletic equipment, the surface, drop-mass and surface interaction, athletic apparel and surface interaction, athletic equipment and surface interaction, or combinations thereof.

5. The system of claim 1, wherein the one or more replaceable impact interfaces represent different athlete- or person-to-surface interaction scenarios.

6. The system of claim 1, wherein the one or more replaceable impact interfaces represent different object- or simulated object-to-surface interaction scenarios.

7. The system of claim 1, wherein a first drop-mass can be replaced by a second drop-mass, and wherein the second drop-mass is not configured to attach or detach replaceable impact interfaces.

8. The system of claim 1, wherein the deceleration data test results include time acceleration history at 10,000 Hz minimum sampling frequency.

9. The system of claim 1, wherein the system measures time acceleration history at 10,000 Hz minimum sampling frequency.

10. The system of claim 1, wherein metrics of one or more of pressure, force, displacement, rebound, impulse, and power, are calculated from an acceleration-vs-time impact time history.

11. The system of claim 1, wherein the drop-mass assembly further comprises the one or more sensors.

12. The system of claim 1, wherein the one or more sensors are one or more accelerometers.

13. The system of claim 1, further comprising a user interface capable of generating or presenting one or more of data, data analysis, or data interpretation, simultaneously or sequentially.

14. The system of claim 1, wherein the one or more processors, one or more controllers, or both, are located remotely from the drop assembly.

15. The system of claim 1, wherein the one or more processors, the one or more controllers, or both, are capable of communicating with a remote electronic device.

16. The system of claim 1, wherein a deceleration of the drop-mass is measured to characterize an interaction of the drop-mass with a surface, or wherein the deceleration of the drop-mass is measured to characterize inertial effects of the drop-mass during use.

17. The system of claim 1, wherein the one or more sensors sense energy absorption and rebound/return through measurement of deceleration of the drop-mass dropped onto a surface.

18. The system of claim 1, wherein the system is capable of providing one or more of the following capabilities:
(a) compile the one or more of, deceleration data test results, force data test results, motion data test results, or acceleration data test results, and display them through a user interface;
(b) compare the one or more of, deceleration data test results, force data test results, motion data test results, or acceleration data test results, against hard-coded or server-based scientifically-determined baseline data to score the one or more of, deceleration data test results, force data test results, motion data test results, or acceleration data test results;
(c) compile and analyze force/torque test results with other test result streams and display to a user;
(d) retrieve historical results from tests and compare with baseline hard-coded data or compare the retrieved historical results with the one or more of, deceleration data test results, force data test results, motion data test results, or acceleration data test results;
(e) evaluate and score geographical consistency of a surface by registering more than one test with location using a Global Positioning System and analyzing test results from multiple locations using correlation, coefficient of variation, standard error, standard deviation, or combinations thereof, to assess variability;
(f) score or rank a surface or surfaces by analyzing a mechanical response of different drop-masses;
(g) flag or recommend localized intervention if a surface may be dangerous; and/or
(h) collect, register, synchronize, retrieve, and/or analyze metadata related to a surface.

19. The system of claim 1, wherein the one or more replaceable impact interfaces are operative to simulate or mimic a shoe, a shoe sole, a cleat, a cleated shoe, a shoe comprising cleats, a helmet, sports equipment, or sports apparel.

20. A system comprising:
a drop assembly comprising a drop-mass for dropping and imparting a force or forces to a surface, the drop-mass configured to directly or indirectly attach to and detach from a plurality of replaceable impact interfaces, wherein dropping a selected replaceable impact interface of the plurality of replaceable impact interfaces as attached to the drop-mass onto a surface is operative to simulate a real-world object interacting with the surface;
a processor, and a computer-readable medium having executable instructions stored thereon, such that, when the executable instructions are executed by the processor, the processor is operative to predict, simulate, or both, interaction dynamics between the surface and the real-world object.

21. The system of claim 20, wherein the object is a shoe, a shoe sole, a cleat, a cleated shoe, a shoe comprising cleats, a helmet, sports equipment, or sports apparel.

22. The system of claim 20, wherein the predictions, simulations, or both, are used to estimate or predict a safety and/or performance measurement, calculation, or score, of the surface, a safety and/or performance measurement, calculation, or score, of the real-world object, or both.

23. The system of claim 20, wherein the predictions, stimulations, or both, are based completely or partially on comparative data resulting from one or more human interacting with a same or a similar surface as the surface onto which the selected replaceable impact interface as attached to the drop-mass is dropped.

24. The system of claim 20, wherein the interaction dynamics include metrics of one or more of, force or forces, deceleration force or forces, traction, shear force or forces, pressure, displacement, rebound, stiffness, shear traction, torque, impulse, or power.

25. The system of claim 20, wherein the interaction dynamics include metrics of one or more of: force or forces in three dimensions, including normal force and/or shear force in x and/or y dimensions; rate of force change; total momentum change; traction; impact surface stress or pressure; impactor displacement; rebound; impulse; energy deposition; energy restitution; or power.

26. The system of claim 20, wherein an angle of the drop-mass dropped onto the surface is adjustable.

27. The system of claim 20, wherein the drop assembly further comprises a track, wherein the drop-mass is slidably connected to the track, wherein the track is adjustable, the adjustability operative to change or adjust an angle at which the drop-mass is dropped onto the surface.

28. The system of claim 27, wherein the drop assembly, the track, or both, can be adjusted such that an angle at which the drop-mass is dropped onto the surface can be changed or adjusted.
